(19) United States
(12) Patent Application Publication
(10) Pub. No.: US 2008/0255431 A1
(43) Pub. Date: Oct. 16, 2008

(54) Early Detection and Warning of Harmful Agents

(75) Inventors: Mendy Erad, Los Altos, CA (US); Yosi Shacham-Diamand, Zikhron-Yaakov (IL); Shimshon Belkin, Kiryat Ono (IL); Arthur Rabner, Natania (IL); Yariv Erad, Kidron (IL); Rami Pedahzur, Harei Yehuda (IL); Yehuda Yarmut, Moshava Yokneam (IL)

Correspondence Address: Martin D. Moynihan;PRTSI, P.O.Box 16446, Arlington, VA, 22215, US

(73) Assignee: Ramot At Tel Aviv Univeristy Ltd., Tel-Aviv (IL)

(21) Appl. No.: 11/659,644

(22) PCT Filed: Aug. 4, 2005
(86) PCT No.: PCT/IL05/00844

§ 371 Date: Feb. 12, 2008

Related U.S. Application Data
(60) Provisional application No. 60/599,033, filed on Aug. 6, 2004.
(60) Provisional application No. 60/625,099, filed on Nov. 5, 2004.

(51) Int. Cl. A61B 5/00; G08B 21/00

(52) U.S. Cl. 600/301; 340/540

(57) ABSTRACT

A method of detecting the presence of harmful agents in the environment is disclosed. The method comprises: providing a plurality of portable agent detectors, placing the detectors on vectors in the environment, connecting the detectors over a communication network, and, upon detection of an agent by a first detector, signaling detectors identified as being nearby to carry out corresponding agent detection tests, thereby to confirm or localize an initial agent detection.

### FIELD AND BACKGROUND OF THE INVENTION

The present invention relates to harmful agents detection and, more particularly, to early detection and warning of presence or diffusion of harmful agents, such as, but not limited to, chemical, biological or radioactive (CBR) agents.

Spread of harmful agents presents a major concern due to the non-local nature of the spread whereby the source of such a threat may be relatively localized, but its effect can appear in many other locations.

Typically, a large time delay occurs between the occurrence of a harmful agent incident, and the time at which the appropriate authorities are able to conclude that a threat is underway. For example, when a region, populated by a particular community, becomes contaminated, e.g., by a cargo spill or by a deliberate act of terrorism, a certain period of time lapses before the contamination or the effect thereof is noticed.

The time delay is either due to the nature of the harmful agent (e.g., gestation period of a biological agent, or artificial delay), or because of a failure in concluding that an observed pattern of events is indicative of the harmful agent incident. It is recognized that if the time delay to detection is long, the incident is likely to be aggravated because the contamination may diffuse to a neighboring population.

Harmful agents may spread to large areas in many ways and many combinations of different ways, such as transport in a medium (e.g., air, surface water, groundwater, soil), transport in via a vehicle (missiles, population movement) reproduction, multiplication, bioaccumulation and the like. This diversity of options makes the propagation patterns of harmful agents almost unpredictable. Unlike conventional bombs, for which the range of damage is limited even if an explosion occurs in a heavily populated location, the damage caused by harmful agent attack can spread, grow with time and cover huge, and in extreme cases, almost unlimited areas.

An utmost pressing problem involving spread of harmful agents is that of early detection and warning. In an era where harmful agent attacks at one or more locations either globally or within a country are possible, it is desirable to have a surveillance system capable of detecting and locating the attack at an early stage. The first priority in the management of harmful agent release events involves detecting that harmful agents have been released, and warning the appropriate authorities of the event. Once the identity and physical properties of the harmful agent that has been released are ascertained, effective measures, such as definition of outer perimeter, evacuation plans, medical treatment, initiation of decontamination procedures, or formulation of neutralization plans, can be taken. Whether the release occurs as a result of enemy activity or as a result of an accident in a domestic facility, a prompt detection is crucial to minimize injury and loss of life.

Known in the art are monitoring devices which are designed to be placed in a particular vulnerable medium, such as a water reservoir or an air condition system. Such monitoring devices can only alert when a harmful agent incident occurs in the in the immediate vicinity of the devices. Threat scenarios, however, are unpredictable in nature and incidents may occur simultaneously in more than one place and/or more than one medium. In particular, such monitoring devices are practically useless for alerting of a silent or even explosive release of a harmful agent in an open environment.

U.S. Pat. No. 6,293,861 discloses a building protection system responsive to the release of airborne agents both outside and inside of a building. An array of sensors surrounding the perimeter of the building is triggered to provide an indication when an external release has occurred. Upon initial detection of a release, a central processor connected to receive a release signal from the sensor shuts down all external air exchanges for the building and activates an over-pressure system for the building interior to insure that contaminated external air does not enter. Exterior sampling inlets are monitored to determine if high agent concentrations exist as a confirmation of the indication given by the perimeter sensors. When an internal attack occurs in the entrance area of the building, an internal sensor transmits an appropriate signal to the central processor, which closes off the entrance area, exhausts air from the closed area through a filter and activates the over-pressure system. Sampling inlets connected in various areas within the building monitor these areas to determine whether they have been contaminated and the concentration and type of contaminating agent. The processor can activate a decontaminant spray system to decontaminate the contaminated areas.

The above system can be typically employed in particular buildings, such as key military sites, which are equipped or designed well in advance to deal with the use of harmful agents. The built-in fixed sensors, which are generally limited to sensing one area of the building, may be too expensive to be placed in all desired areas of the building. Other facilities, such as hotels, department stores, shopping malls and the like, are more susceptible to harmful agents, lacking even the aforementioned fixed sensors.

An additional device, developed by Oak Ridge National Laboratory, Tennessee, USA, includes a biochip with several types of bioreceptors, which identify biological warfare agents. The biochip is combined with an electronic microchip having phototransistors which read fluorescence emitted from the bioreceptors.

U.S. Pat. No. 6,411,207 discloses a personal alert device, which includes one or more sensors for detecting a possible physical threat to the user. The sensors are connected to a processor which generates an alert message relating to a recommended course of action for the user to avoid the possible physical threat.

U.S. Pat. No. 6,701,772 discloses a system for detecting harmful agents in buildings. The system includes a moving detector which traverses spaces in the buildings, detects presence of harmful agents and transmits data to a receiver. The data includes both the type of harmful agent and the location at which the agent was detected.

U.S. Pat. No. 6,710,711 discloses a method of identifying hazards occurring within an area by combining syndromic data with modeling and simulation operation. The actual syndromic data is compared with the simulation results to determine whether or not the syndromic data correlates with the simulation results.

The above prior art attempts have been exclusively directed to provide a local scale detection of harmful agents, typically in a predetermined site, by employing either a fixed arrangement of sensors, or a self propelled detector to locally cover a confined area. However, because the technological and financial problems associated with adaptation of known techniques a global scale, the harmful agent detection solutions provided by prior art are far from being satisfactory.

There is thus a widely recognized need for, and it would be highly advantageous to have method and means for early detection and warning of presence or diffusion of harmful agents, devoid of the above limitations.

### SUMMARY OF THE INVENTION

According to one aspect of the present invention there is provided a method of determining presence or absence of harmful agents in the environment, comprising: providing a plurality of portable agent detectors having an operative mode and a non-operative mode; selecting non-operative modes for at least a portion of the plurality of detectors, and placing the plurality of detectors on vectors in the environment; connecting the plurality of detectors over a communication network having a plurality of base stations; activating detectors associated with at least one base station to carry out agent detection tests; and receiving information from the activated detectors, thereby determining presence or absence of harmful agent in a region defined by the activated detectors.

According to further features in preferred embodiments of the invention described below, the communication network comprises a cellular communication network.

According to still further features in the described preferred embodiments the detectors are integrated with cellular telephones.

According to still further features in the described preferred embodiments the method further comprises identifying clustering detection events received from the activated detectors.

According to still further features in the described preferred embodiments the method further comprises obtaining atmospheric conditions at the region and using the atmospheric condition to predict a propagation path of the agent.

According to still further features in the described preferred embodiments the method further comprises activating detectors identified as localized on or near the propagation path.

According to another aspect of the present invention there is provided a method of detecting the presence of harmful agents in the environment comprising: providing a plurality of portable agent detectors, placing the detectors on vectors in the environment, connecting the detectors over a communication network, upon detection of an agent by a first detector, signaling detectors identified as being nearby to carry out corresponding agent detection tests, thereby to confirm or localize an initial agent detection.

According to still further features in the described preferred embodiments the method further comprises monitoring the detectors at a central location via the communication network.

According to still further features in the described preferred embodiments the method further comprises alerting vectors identified as being nearby to the first detector.

According to yet another aspect of the present invention there is provided a method of detecting the presence of harmful agents in the environment comprising: providing a plurality of portable agent detectors able to generate a detection event in the presence of any of the harmful agents, placing the detectors on vectors in the environment, connecting the detectors over a communication network, at a central location monitoring the detectors to identify clustering of the detection events, the clustering being to confirm or localize an initial agent detection.

According to further features in preferred embodiments of the invention described below, the method further comprises, upon a detection event generated by a first detector, signaling detectors identified as being nearby to the first detector, to carry out corresponding agent detection tests.

According to still further features in the described preferred embodiments the method further comprises, upon a detection event generated by a first detector, alerting vectors identified as being nearby to the first detector.

According to still another aspect of the present invention there is provided a method of detecting the presence of harmful agents, comprising: providing a plurality of portable agent detectors able to generate a detection event in the presence of any of the harmful agents, associating the detectors with cellular communication units to allow central monitoring of the detectors, and placing the detectors and cellular communication units on vectors in the environment.

According to still further features in the described preferred embodiments the method further comprises associating the detectors with positioning units, and monitoring locations of individual detectors using a positioning network.

According to still further features in the described preferred embodiments the method further comprises upon a detection event generated by a first detector, signaling detectors identified as being nearby to the first detector, to carry out corresponding agent detection tests.

According to still further features in the described preferred embodiments the signaling the nearby detectors comprises transmitting instructions to respective vectors to locally activate the nearby detectors.

According to still further features in the described preferred embodiments the signaling the nearby detectors comprises a remote activation, performed at the central location.

According to still further features in the described preferred embodiments the method further comprises obtaining atmospheric conditions at a location of the first detector and using the atmospheric condition to predict a propagation path of the agent.

According to still further features in the described preferred embodiments the method further comprises alerting vectors identified as localized on or near the propagation path.

According to still further features in the described preferred embodiments the vectors are mammals.

According to still further features in the described preferred embodiments the method further comprises measuring vital signs of the mammals.

According to an additional aspect of the present invention there is provided a method for analyzing a diffuse signal from an environment comprising: releasing a plurality of signal detectors into the environment, each signal detector being able to generate a detection event in the presence of the diffuse signal; monitoring each of the signal detectors over a communication network for the detection events and for current location data of the signal detectors; and identifying clustering of the detection events as an indication of the presence of the diffuse signal.

According to further features in preferred embodiments of the invention described below, the method further comprises, upon a detection event generated by a first detector, signaling detectors identified as being nearby to the first detector, to carry out corresponding detection tests.

According to still further features in the described preferred embodiments the method further comprises, upon a detection event generated by a first detector, generating alert signals and transmitting the alert signals to detectors identified as being nearby to the first detector.

According to still further features in the described preferred embodiments the signaling comprises transmitting instructions to vectors carrying the nearby detectors to locally activate the nearby detectors.

According to still further features in the described preferred embodiments the signaling comprises remotely activating the nearby detectors.

According to yet an additional aspect of the present invention there is provided a method for analyzing a diffuse signal from an environment comprising: releasing a plurality of signal detectors into the environment, each signal detector being able to generate a detection event in the presence of the diffuse signal; monitoring each of the signal detectors over a communication network for the detection events and for current location data of the signal detectors; upon identifying a detection event, checking with other nearby detectors for confirmation of the diffuse signal.

According to further features in preferred embodiments of the invention described below, the checking with the nearby detectors comprises transmitting instructions to vectors carrying the nearby detectors to locally activate the nearby detectors.

According to still further features in the described preferred embodiments the checking with the nearby detectors comprises remotely activating the nearby detectors.

According to still further features in the described preferred embodiments the method further comprises identifying clustering of the detection events.

According to still further features in the described preferred embodiments the method further comprises obtaining atmospheric conditions at a location of at least one detector and using the atmospheric condition to predict a propagation path of the diffuse signal.

According to still further features in the described preferred embodiments the method further comprises generating alert signals and transmitting the alert signals to detectors identified as localized on or near the propagation path.

According to still an additional aspect of the present invention there is provided a portable agent detector for releasing into an environment to detect harmful agents released into the environment, the agent detector comprising: a removable sensing cassette comprising sensing wetware; and a processing unit for processing signals from the sensing cassette.

According to still further features in the described preferred embodiments the portable agent detector is identifiable by an identification code, and the communication unit is operable to transmit the identification code over the communication network.

According to still further features in the described preferred embodiments the portable agent detector further comprises an automatic sampling unit for automatically sampling fluids from the environment, and transferring the fluids to the sensing cassette.

According to a further aspect of the present invention there is provided a distributed detection system for detection of the presence of harmful agents in an environment, comprising: a central monitoring unit; and a plurality of portable agent detectors enabled for communication with the central monitoring unit and configured for producing detection events in the presence of the harmful agents and communicating the detection events to the central monitoring unit; the portable agent detectors being mounted on mobile vectors for release into the environment.

According to still further features in the described preferred embodiments at least one of the plurality of portable agent detectors comprises: a sensing unit having an operative mode and a non-operative mode; an activation unit being in communication with a central location, for selecting between the operative mode and the non-operative mode; and a processing unit for processing signals from the sensing unit.

According to still further features in the described preferred embodiments at least one of the plurality of portable agent detectors further comprises a user interface, wherein the activation unit is controllable by the user interface.

According to still further features in the described preferred embodiments at least one of the plurality of portable agent detectors further comprises a user display.

According to still further features in the described preferred embodiments at least one of the plurality of portable agent detectors further comprises a supplementary sensing unit capable of continuously monitoring environmental conditions, and generating a signal to the activation unit to activate the portable agent detector when the environmental conditions meet a predetermined set of criteria.

According to still further features in the described preferred embodiments at least one of the plurality of portable agent detectors further comprises a communication unit, for transmitting signals representing presence, level or absence of harmful agents over a communication network.

According to still further features in the described preferred embodiments at least one of the plurality of portable agent detectors is identifiable by an identification code, and each communication unit is operable to transmit a respective identification code over the communication network.

According to still further features in the described preferred embodiments at least one of the plurality of portable agent detectors further comprises a positioning unit, for determining a location of the portable agent detector, and each communication unit is operable to transmit a respective location over the communication network.

According to still further features in the described preferred embodiments at least one of the plurality of portable agent detectors further comprises detection hardware.

According to still further features in the described preferred embodiments at least one of the plurality of portable agent detectors further comprises vital signs measuring unit for measuring vital signs of a mammal carrying the portable agent detector.

According to still further features in the described preferred embodiments at least one of the plurality of portable agent detectors further comprises an atmospheric condition measuring unit for measuring at least one atmospheric condition.

According to still further features in the described preferred embodiments at least one of the plurality of portable agent detectors further comprises an automatic sampling unit for automatically sampling fluids from the environment, and transferring the fluids to the sensing unit.

According to still further features in the described preferred embodiments at least one of the plurality of portable agent detectors further comprises an armored encapsulation.

According to still further features in the described preferred embodiments at least one of the plurality of portable agent detectors further comprises an image capturing unit.

According to still further features in the described preferred embodiments at least one of the plurality of portable agent detectors further comprises an input-output audio unit.

According to yet a further aspect of the present invention there is provided a portable agent detector for releasing into an environment to detect harmful agents released into the environment, the agent detector comprising: a sensing unit having an operative mode and a non-operative mode; an activation unit being in communication with a central location, for selecting between the operative mode and the non-operative mode; and a processing unit for processing signals from the sensing unit.

According to further features in preferred embodiments of the invention described below, the portable agent detector further comprises a communication unit, for transmitting signals representing presence, level or absence of harmful agents over a communication network.

According to still further features in the described preferred embodiments the portable agent detector further comprises a positioning unit, for determining a location of the agent detector, wherein the communication unit is operable to transmit the location over the communication network.

According to still further features in the described preferred embodiments the portable agent detector further comprises an atmospheric condition measuring unit for measuring at least one atmospheric condition.

According to still a further aspect of the present invention there is provided a portable agent detector for releasing into an environment to detect harmful agents released into the environment, the agent detector comprising: a sensing unit for detecting presence or absence of harmful agents; a processing unit for processing signals from the sensing unit; a positioning unit for determining a location of the agent detector; and a communication unit for transmitting the location and signals representing presence or absence of harmful agents over a communication network.

According to further features in preferred embodiments of the invention described below, the portable agent detector further comprises an atmospheric condition measuring unit for measuring at least one atmospheric condition, and further wherein the communication unit is operable to transmit the at least one atmospheric condition over the communication network.

According to still further features in the described preferred embodiments the portable agent detector further comprises an activation unit for activating or selecting an operational mode of the portable agent detector.

According to still further features in the described preferred embodiments the portable agent detector further comprises a user interface, wherein the activation unit is controllable by the user interface.

According to still further features in the described preferred embodiments the user interface is designed and configured to generate at least one sensible signal being indicative of an operative status and/or impermeability level of the portable agent detector.

According to still further features in the described preferred embodiments the activation unit is in communication with a central location and being controllable thereby, hence allowing a remote activation or operational mode selection of the portable agent detector.

According to still further features in the described preferred embodiments the portable agent detector further comprises a user display.

According to still further features in the described preferred embodiments the portable agent detector further comprises a supplementary sensing unit capable of continuously monitoring environmental conditions, and generating a signal to the activation unit to activate the portable agent detector when the environmental conditions meet a predetermined set of criteria.

According to still further features in the described preferred embodiments the portable agent detector further comprises detection hardware.

According to still further features in the described preferred embodiments the portable agent detector further comprises vital signs measuring unit for measuring vital signs of a mammal carrying the portable agent detector.

According to still further features in the described preferred embodiments the vital signs measuring unit is designed and configured to generate a signal to the activation unit to activate the portable agent detector when the vital signs of the mammal meet a predetermined set of criteria.

According to still further features in the described preferred embodiments the portable agent detector further comprises an automatic sampling unit for automatically sampling fluids from the environment, and transferring the fluids to the sensing unit.

According to still further features in the described preferred embodiments the portable agent detector further comprises an armored encapsulation.

According to still further features in the described preferred embodiments the portable agent detector further comprises an image capturing unit.

According to still further features in the described preferred embodiments the portable agent detector further comprises an input-output audio unit.

According to still a further aspect of the present invention there is provided a portable vital signs detector to detect vital signs of a mammal the portable vital signs detector comprising: a sensing unit for detecting the vital signs of the mammal; a processing unit for processing signals from the sensing unit; a positioning unit for determining a location of the vital signs detector; and a communication unit for transmitting the location and signals representing the vital signs.

According to further features in preferred embodiments of the invention described below, the portable vital signs detector further comprises an activation unit for activating or selecting an operational mode of the portable vital signs detector.

According to still further features in the described preferred embodiments the portable vital signs detector further comprises a user interface, wherein the activation unit is controllable by the user interface.

According to still further features in the described preferred embodiments the activation unit is in communication with a central location and being controllable thereby, hence allowing a remote activation or operational mode selection of the portable vital signs detector.

According to still further features in the described preferred embodiments the portable vital signs detector further comprises a user display.

According to still further features in the described preferred embodiments the portable vital signs detector further comprises an atmospheric condition measuring unit for measuring at least one atmospheric condition.

According to still further features in the described preferred embodiments the portable vital signs detector further comprises an image capturing unit.

According to still further features in the described preferred embodiments the portable vital signs detector further comprises an input-output audio unit.

According to still a further aspect of the present invention there is provided a personal accessory device having an agent detector to detect harmful agents released into the environment, the agent detector comprising: a sensing unit having an operative mode and a non-operative mode; an activation unit being in communication with a central location, for selecting between the operative mode and the non-operative mode; and a processing unit for processing signals from the sensing unit.

According to further features in preferred embodiments of the invention described below, the activation unit is capable of deactivating any functioning unit of the personal accessory device other than the sensing unit and the processing unit.

According to still further features in the described preferred embodiments the user display is in communication with the central location and being controllable thereby, hence allowing the central location to communicate, at least unilaterally, with a user.

According to still further features in the described preferred embodiments the personal accessory device further comprises a communication unit, for transmitting signals representing presence, level or absence of harmful agents over a communication network.

According to still further features in the described preferred embodiments the personal accessory device is identifiable by an identification code, and the communication unit is operable to transmit the identification code over the communication network.

According to still further features in the described preferred embodiments the personal accessory device further comprises a positioning unit, for determining a location of the agent detector, wherein the communication unit is operable to transmit the location over the communication network.

According to still further features in the described preferred embodiments the personal accessory device further comprises an atmospheric condition measuring unit for measuring at least one atmospheric condition.

According to still a further aspect of the present invention there is provided a personal accessory device having an agent detector to detect harmful agents released into the environment, the agent detector comprising: a sensing unit for detecting presence or absence of harmful agents; a processing unit for processing signals from the sensing unit; a positioning unit for determining a location of the agent detector; and a communication unit for transmitting the location and signals representing presence or absence of harmful agents over a communication network.

According to further features in preferred embodiments of the invention described below, the personal accessory device further comprises an atmospheric condition measuring unit for measuring at least one atmospheric condition, and further wherein the communication unit is operable to transmit the at least one atmospheric condition over the communication network.

According to still further features in the described preferred embodiments the sensing unit is environmentally sealable.

According to still further features in the described preferred embodiments the sensing unit and the processing unit are designed and constructed to provide detection information within predetermined and variable time periods.

According to still further features in the described preferred embodiments the personal accessory device further comprises a user interface, wherein the activation unit is controllable by the user interface.

According to still further features in the described preferred embodiments the personal accessory device further comprises a user display.

According to still further features in the described preferred embodiments the personal accessory device further comprises a supplementary sensing unit capable of continuously monitoring environmental conditions, and generating a signal to the activation unit to activate the agent detector when the environmental conditions meet a predetermined set of criteria.

According to still further features in the described preferred embodiments the personal accessory device further comprises detection hardware.

According to still further features in the described preferred embodiments the personal accessory device further comprises vital signs measuring unit for measuring vital signs of a mammal carrying the agent detector.

According to still further features in the described preferred embodiments the personal accessory device further comprises an automatic sampling unit for automatically sampling fluids from the environment, and transferring the fluids to the sensing unit.

According to still further features in the described preferred embodiments the personal accessory device further comprises an armored encapsulation.

According to still further features in the described preferred embodiments the personal accessory device further comprises an image capturing unit.

According to still further features in the described preferred embodiments the personal accessory device further comprises an input-output audio unit.

According to still a further aspect of the present invention there is provided a portable detection kit for releasing into an environment to detect harmful agents released into the environment, the portable detection kit comprising: at least one sampling device for selectively sampling environmental materials; and an agent detector having a removable sensing cassette comprising sensing wetware, and a processing unit for processing signals from the sensing wetware.

According to further features in preferred embodiments of the invention described below, the at least one sampling device is reusable.

According to still further features in the described preferred embodiments the at least one sampling device is adapted to sample fluids.

According to still further features in the described preferred embodiments the at least one sampling device is adapted to sample solids.

According to still further features in the described preferred embodiments the at least one sampling device comprises a syringe and a syringe needle.

According to still further features in the described preferred embodiments the at least one sampling device is adapted to continuously sample the environmental materials.

According to still further features in the described preferred embodiments the at least one sampling device comprises a container and at least one treating element for treating the environmental materials in the container.

According to still further features in the described preferred embodiments the portable detection kit further comprises at least one medicament.

According to still further features in the described preferred embodiments the portable detection kit further comprises an injector for injecting the at least one medicament.

According to still further features in the described preferred embodiments the removable sensing cassette is environmentally sealable.

According to still further features in the described preferred embodiments the sensing cassette and the processing unit are designed and constructed to provide detection information within predetermined and variable time periods.

According to still further features in the described preferred embodiments the portable detection kit further comprises an activation unit for activating or selecting an operational mode of the agent detector.

According to still further features in the described preferred embodiments the portable detection kit further comprises a user interface, wherein the activation unit is controllable by the user interface.

According to still further features in the described preferred embodiments the user interface is designed and configured to generate at least one sensible signal being indicative of presence, level or absence of harmful agents.

According to still further features in the described preferred embodiments a level of the at least one sensible signal is selected to allow sensation of the at least one sensible signal at large distances.

According to still further features in the described preferred embodiments the user interface is designed and configured to generate at least one sensible signal being indicative of an operative status and/or impermeability level of the agent detector According to still further features in the described preferred embodiments the activation unit is in communication with a central location and being controllable thereby, hence allowing a remote activation or operational mode selection of the agent detector.

According to still further features in the described preferred embodiments the portable detection kit further comprises a user display.

According to still further features in the described preferred embodiments the user display is in communication with a central location and being controllable thereby, hence allowing the central location to communicate, at least unilaterally, with a user.

According to still further features in the described preferred embodiments the portable detection kit further comprises a supplementary sensing unit capable of continuously monitoring environmental conditions, and generating a signal to the activation unit to activate the agent detector when the environmental conditions meet a predetermined set of criteria.

According to still further features in the described preferred embodiments the predetermined set of criteria comprises preliminary detection of a potentially harmful agent.

According to still further features in the described preferred embodiments the portable detection kit further comprises a communication unit, for transmitting signals representing presence, level or absence of harmful agents over a communication network.

According to still further features in the described preferred embodiments the portable detection kit is identifiable by an identification code, and further wherein the communication unit is operable to transmit the identification code over the communication network.

According to still further features in the described preferred embodiments the agent detector further comprises detection hardware.

According to still further features in the described preferred embodiments the detection hardware comprises optical detection hardware.

According to still further features in the described preferred embodiments the agent detector further comprises vital signs measuring unit for measuring vital signs of a mammal carrying the agent detector.

According to still further features in the described preferred embodiments the vital signs measuring unit is designed and configured to generate a signal to the activation unit to activate the agent detector when the vital signs of the mammal meet a predetermined set of criteria.

According to still further features in the described preferred embodiments the agent detector further comprises an atmospheric condition measuring unit for measuring at least one atmospheric condition.

According to still further features in the described preferred embodiments the agent detector further comprises an automatic sampling unit for automatically sampling fluids from the environment, and transferring the fluids to the sensing cassette.

According to still further features in the described preferred embodiments the agent detector further comprises an armored encapsulation.

According to still further features in the described preferred embodiments the armored encapsulation is at least partially impermeable and capable of withstanding extreme thermal and/or mechanical conditions.

According to still further features in the described preferred embodiments the agent detector further comprises an image capturing unit.

According to still further features in the described preferred embodiments the agent detector further comprises an input-output audio unit.

According to still further features in the described preferred embodiments the communication unit is supplemented with at least one communication protocol, tangibly embodied in a readable memory, the at least one communication protocol being configured to allow a takeover of the communication network.

According to still a further aspect of the present invention there is provided a sampling device for selectively sampling environmental materials, the sampling device comprising a sampling element, a container and at least one treating element for treating the environmental materials in the container.

According to still further features in the described preferred embodiments at least one of the sampling element, the container and the treating element is reusable.

According to still further features in the described preferred embodiments sampling element is adapted to sample fluids.

According to still further features in the described preferred embodiments the sampling element is adapted to sample solids.

According to still further features in the described preferred embodiments sampling element comprises a syringe and a syringe needle.

According to still further features in the described preferred embodiments the sampling element is adapted to continuously sample the environmental materials.

According to still further features in the described preferred embodiments the at least one treating element is selected from the group consisting of a filter, an enriching unit, an elution unit, a heating unit, an irradiation unit, a labeling unit, a separating column and a sorter.

According to still further features in the described preferred embodiments the at least one treating element comprises at least one biological material.

According to still further features in the described preferred embodiments the at least one treating element is designed and constructed to perform a nucleic acid amplification procedure.

The present invention successfully addresses the shortcomings of the presently known configurations by providing a system, a detector, a kit and methods for early detection and warning of presence or diffusion of harmful agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a flowchart diagram of a method of detecting the presence of harmful agents in the environment, according to a preferred embodiment of the present invention.

FIG. 2 is a schematic illustration of a portable agent detector for releasing into an environment to detect harmful agents, according to a preferred embodiment of the present invention.

FIG. 3 is a schematic illustration of a portable detection kit for releasing into an environment to detect harmful agents, according to a preferred embodiment of the present invention.

FIGS. 4a-c are flowchart diagrams of several general treatment scenarios, applied prior to the application of the detector, according to a preferred embodiment of the present invention.

FIGS. 5a-c are flowchart diagrams of treatment scenarios, applied for the cases of air (FIG. 5a), liquid (FIG. 5b) and surface (FIG. 5c) sampling, according to a preferred embodiment of the present invention.

FIG. 6 is a schematic illustration of a distributed detection system, for detection of presence of harmful agents in the environment according to a preferred embodiment of the present invention.

### DESCRIPTION OF THE PREFERRED EMBODIMENTS

The present embodiments are of methods of detecting the presence of harmful agents which can be chemical, biological or radioactive agents. The present embodiments can be used for analyzing diffuse signals, generated, e.g., by propagation of harmful agents in the environment. The present embodiments are further of portable agent detectors and portable detection kits, which can be locally utilized to detect the presence of harmful agents. Additionally, the present embodiments are of a distributed system employing the agent detectors and/or the portable detection kits.

The principles and operation of the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As an advantage, the present embodiments provide various early warnings of a potential unconventional attack, such as biological, chemical or radioactive attack. The present embodiments are effective against deliberate events (e.g., biological, chemical or radioactive terrorism), accidents causing release of harmful agents (e.g., cargo spills) and natural events (e.g., epidemics).

As will be appreciated by one of ordinary skill in the art, the present embodiments provide practical, inexpensive and easily applicable solution to the problem of harmful agent detection.

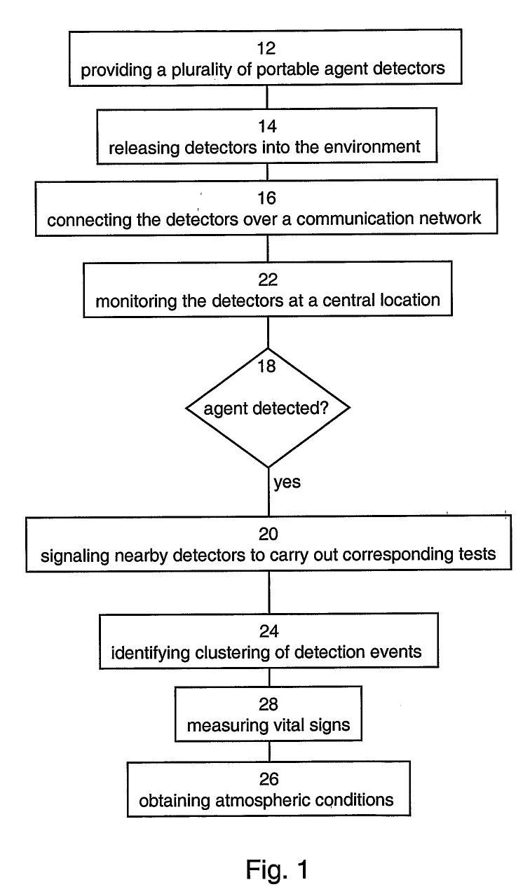

Referring now to the drawings, FIG. 1 is a flowchart diagram of a method of detecting the presence of harmful agents in the environment. It is to be understood, that unless otherwise defined, the method steps described hereinbelow can be executed either contemporaneously or sequentially in any combination or order of execution. Specifically, neither the ordering of the flowchart of FIG. 1, nor the numerals designating its various blocks are to be considered as limiting. For example, two or more method steps, appearing in the description or in the flowchart of FIG. 1 in a particular order, can be executed in a different order (e.g., a reverse order) or substantially contemporaneously.

Hence, in a first step of the method, designated by Block 12, a plurality of portable agent detectors is provided. The detectors preferably detect biological, chemical and/or radioactive agents, in any form, including, without limitation fluid (gas or liquid), solid (particulates, aggregates or contaminated surfaces), vapor, droplet and the like. According to various exemplary embodiments of the present invention, the detectors are capable of detecting and/or identifying any of the above agents. In other words, the detectors preferably provide (i) indication whether or not an agent is present in the environment, without identifying the agent; and/or (ii) a certain level of identification of the detected agent, which certain level of identification can be either a general identification (e.g., biological/chemical/radioactive) or a specific identification (e.g., specific composition). In any event, once any of the detectors detect or identify presence of a harmful agent a signal is generated as further detailed hereinunder.

Many types of agent detectors are contemplated, for example, wetware based detectors (e.g., arrays of immobilized reporter cells) or hardware based detectors (e.g., electromagnetic radiation emitters, agent-sensitive semiconductors). According to a preferred embodiment of the present invention the detectors simultaneously detect and/or identify more than one harmful agent. Various types of agent detectors which are suitable for the present embodiments are found in Franz L. Dickert et al., “Sensor strategies for microorganism detection—from physical principles to imprinting procedures,” Anal Bioanal Chem (2003) 377:540-549, and U.S. Pat. Nos. 6,411,207, 6,743,581, 6,710,711, 6,679,099, 6,558,626, 6,448,064, 6,197,503 and 6,159,681, the contents of which are hereby incorporated by reference. Also contemplated, is the aforementioned device of Oak Ridge National Laboratory.

Any number of the portable agent detectors can be used, e.g., tens, hundreds, thousands, hundreds of thousands, millions and more. As will be appreciated, larger number detectors can provide coverage of larger areas. Hence, according to a preferred embodiment of the present invention the number of portable agent detectors is comparable with the size of the population occupying the environment of interest.

In another step, designated by Block 14, the detectors are released to the environment, preferably by placing the detectors on vectors in the environment.

As used herein, a “vector” refers to an entity having a self-relocating ability. For example, a “vector” can be a civilian, a law-enforcement officer, a vehicle, an animal and the like.

The portable agent detectors are preferably, but not obligatory, integrated with or mounted on personal accessories, such as, but not limited to, cellular telephones, personal digital assistants, laptops and key holders, which are typically carried by the residents on normal routine. In this embodiment, the vectors are individuals of the population, which, by following their daily routines, span a substantially large detection area.

In an additional step, designated by Block 16 the detectors are connected over a communication network, for example, wireless local area network (WLAN), Wi-Fi® network, Bluetooth® network, cellular network and the like. Preferably, the detectors are connected to more that one communication network to allow operation also in regions not covered by a particular network.

Optionally and preferably, the detectors are supplemented by positioning units to allow them to transmit location data over the network. Many positioning technologies are contemplated. Representative examples include, without limitation global positioning systems, commonly known as GPS, network based positioning, in which the location of the detector is computed by triangulation of its signal between transmission towers, motion-based positioning in which the location is calculated based on the motion parameters of the detector, and cell-identification in which the environment is divided into a plurality of geometric elements and each entry of the detector into a respective element is monitored and recorded.

Thus, according to a preferred embodiment of the present invention the method comprises an optional step, designated by Block 22, in which the detectors are monitored at a central location. The monitoring can be done in terms of detection events as well as in terms of locations. The monitoring can be done either continuously, or at predetermined times as desired. The central location can use the information acquired from the detectors to perform risk analysis and, based on the analysis, to notify the appropriate authorities (decontamination divisions, medical teams, low enforcement, mass communication channels, etc.) of the location of the detected threat.

Another step of the method, designated by decision Block 18 and process Block 20, is preferably executed upon detection event generated by a particular detector. In this step, other detectors, identified as being nearby to the particular detector, are signaled to carry out corresponding agent detection tests, to thereby confirm or localize initial agent detection.

As will be appreciated by one ordinarily skilled in the art, the use of many portable detectors which communicate over the network and interchange detection and, optionally, location data can be used to detect presence, level and location of a threat and to distribute the information, substantially in real-time, both to the appropriate authorities and among the population. The activation of the nearby detectors can be done either by instructing the respective vectors which carry the nearby detectors to locally activate the detectors, or by performing a remote activation at the central location. Additionally, certain vectors can be alerted of the threat and instructed to take the necessary precautions.

It is to be understood that the activation of the detectors can be done, irrespectively whether or not a detection event has been received. According to various exemplary embodiments of the present invention, the detectors (or at least a portion thereof) are distributed in the environment in an inactive mode. The appropriate authorities can then decide, e.g., based on intelligence or other sources of information, to selectively activate detectors which are located in a particular region of interest. This can be done, for example, by transmitting activating signals to detectors associated with one or more base stations of the communication network which cover the region. This embodiment is particularly useful when the detectors are integrated in their inactive mode within cellular telephones carried by the population, whereby each cellular telephone is frequently communicating its nearest cellular base stations, and all the cellular telephones which are associated with the same cellular base station define a region. Activating signals, transmitted through a particular cellular base station, activate only the detectors of the defined region while keeping detectors located in other regions in their inactive mode. It will be appreciated that this can prevent accidental activation and panic in all regions other than the region of interest.

In an additional step, designated by Block 24, clustering of detection events are preferably identified, by combining detection and location information received, e.g., at the central location. The clustering can serve both as confirmation for the presence, level and location of the harmful agent, and to assess the diffusion rate, e.g., by repeating the clustering identification at different instants of time.

Another optional, yet preferred, step of the method, particularly useful when the vectors are human or animals, is designated in Block 28. In this step, vital signs of the human or animal are measured and transmitted over the network. As will be appreciated, a change, typically reduction, in vital signs occurring in a plurality of subjects occupying a substantially limited area, can be indicative of presence of a harmful agent in the area. Hence, the present embodiments can be used to analyze a diffuse signal from the environment, which signal is in the form of event detection of either a harmful agent or an causal effect thereof.

According to a preferred embodiment of the present invention the method may further comprise another step, designated by Block 26, in which atmospheric conditions are obtained, for example, at a particular location in which a detection event was generated. The atmospheric conditions can be used, alone or in combination with other information (e.g., clustering information) to predict a propagation path of the detected agent. The propagation path can also be transmitted over the communication network, for example, for alerting the vectors which are localized on or near the propagation path.

It will be appreciated by one ordinarily skilled in the art that the detection and/or identification capability of the detectors can be exploited also to provide information when the environment is not contaminated. For example, the detectors can be activated, in any of the above scenarios, after a certain region has been decontaminated so as to detect presence or identify type of harmful agents which were not affected by the decontamination procedure. Optionally and preferably, the detectors can provide an “all clear” indication (e.g., by generating a sensible signal or by transmitting the information over the communication network) when no harmful agent was detected, or when the level of presence of the agents is sufficiently low.

According to another aspect of the present invention there is provided a portable agent detector 30 for releasing into an environment to detect harmful agents released into environment.

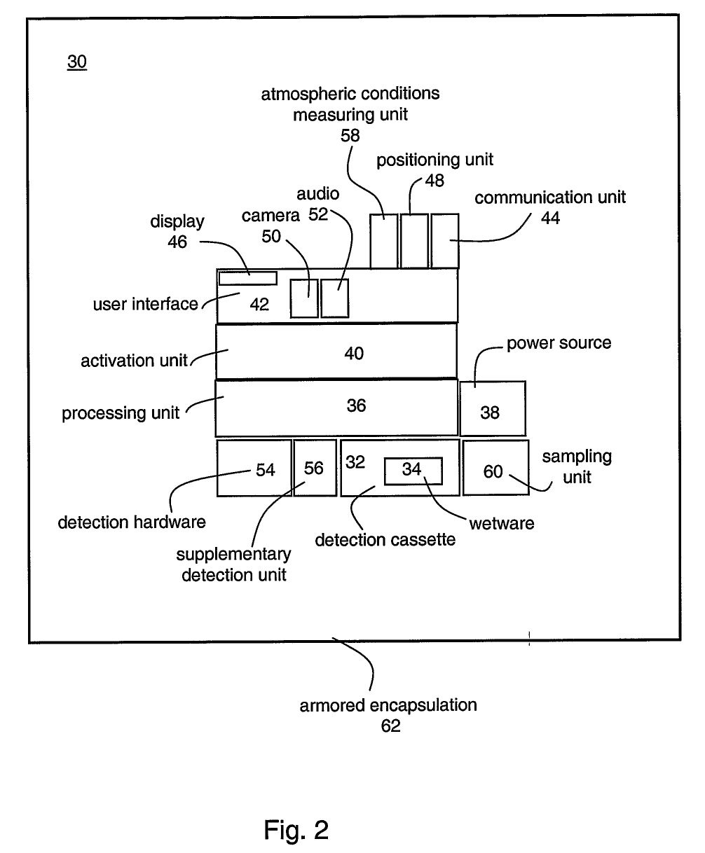

Reference is now made to FIG. 2 which is a schematic illustration of detector 30. In one embodiment, detector 30 comprises a removable sensing cassette 32 having sensing wetware 34, and a processing unit 36 for processing signals from sensing cassette 32.

Sensing cassette 32 is preferably environmentally sealable and capable of detecting biological, chemical and/or radioactive materials, upon activation of detector 30. Sensing cassette 32 can include, for example, arrays of immobilized reporter cells located on a solid matrix. Fluids from the environment can be transferred to sensing cassette 32 by an automatic sampling unit 60, or by any other device as further detailed hereinunder.

The signals generated by sensing cassette 32, can be optical, electrical or acoustic signals. Processing unit 36 may convert the signals as a part of the processing procedure. For example, when the signals are optical or acoustic, they can be converted into electronic signals (e.g., analog or preferably digital) which in turn are analyzed. Processing unit 36 preferably receives signals simultaneously from several areas of cassette 32, for example, in case several types of wetware are placed in different locations in cassette 32.

When the signals generated by wetware 34 are optical, unit 36 can comprise a plurality of addressable elementary units, each being capable of converting optical signals into electrical signals. When an optical signal originating from a particular location in cassette 32 impinges on an elementary unit, an electrical signal is generated. The plurality of electrical signals generated by all the elementary units thus comprises imagery information hence allowing the attribution of each signal to a respective location.

Several types of elementary detection units are contemplated. Representative examples include, without limitation, positive-intrinsic-negative (PIN) photodiodes, avalanche photodiodes, silicon chips, photomultipliers and the like.

According to a preferred embodiment of the present invention detector 30 has a prolonged shelf life, so as to allow accumulation of large number of items of detector 30 to be distributed to the population. Preferably, detector 30 is designed to be stored in a non-operative mode under ambient conditions for prolonged periods of time without loss of functionality. Selected components of detector 30, (e.g., wetware 34) may be stored under lower temperatures (e.g., 0-10° C.) to further extend the shelf life of detector 30.

According to a preferred embodiment of the present invention cassette 32 and processing unit 36 are designed and constructed to provide detection information within predetermined and variable time periods. Specifically, the time frame for detecting a particular agent depends upon the magnitude of its threat. For example, for acute, life threatening agents, the detection time is preferably from a few seconds up to one or a few minutes; for less harmful agents, the detection can be within several (say about 15) minutes. Additionally, detector 30 is preferably capable of providing preliminary detection information at an early time (say within a few seconds), and a final detection information at a later time, depending on the type of threat and detection means.

As used herein the term “about” refers to ±10%.

Optionally and preferably, processing unit 36 is supplemented by an algorithm designed to perform risk assessments based on the information regarding the presence or level of harmful agent in the environment. The risk assessments can be also based on other information, such as, but not limited to, atmospheric conditions, time of the day, season, and the like. According to a preferred embodiment of the present invention processing, based on the risk assessments, unit 36 can also provide the user with specific instructions, e.g., suitable medication or protection means, evacuation routes and the like.

Detector 30 may further comprise other means of detection, e.g., detection hardware 54, which can be, for example, optical detection hardware. Detection hardware 54, is preferably used for short range detection, by emitting an optical signal toward the agent and receiving optical response therefrom. This embodiment is particularly useful for detecting specific toxicants, such as, but not limited to, airborne and/or surface-laden lethal toxic particles, e.g., anthrax and chemical agents. Additionally, detection hardware 54 can be used to provide data regarding air-flow directions.

Detector 30 preferably comprises a power source 38 for supplying energy to its components, e.g., processing unit 36 and other components which may be employed, as further detailed hereinunder. Power source 38 is preferably portable, and can be replaceable or rechargeable, integrated with or being an accessory to detector 30. Representative examples include, without limitation a solar power source, a mobile voltage generator, an electrochemical cell, a traditional secondary (rechargeable) battery, a double layer capacitor, an electrostatic capacitor, an electrochemical capacitor, a thin-film battery (e.g., a lithium cell), a microscopic battery and the like.

Alternatively, power source 38 can be a fixed power source, for example, a power source from a wall socket or a fixed voltage generator. According to a preferred embodiment of the present invention power source 38 can be disconnected from detector 30 without the need to open sealed components (e.g., cassette 34).

The type and size of power source 38 as well as the amount of energy stored therein may vary, depending on the required power and, in some embodiments, on the component in which power source 30 is implemented. Preferably, the life time of power source 30 when detector 30 is not operative is comparable to the shelf life of detector 30. In operative mode, the life time of power source 30 is preferably above 1 hour more preferably above 10 hours most preferably above 100 hours.

Detector 30 preferably comprises an activation unit 40 for activating or selecting an operational mode of the portable agent detector. Activation unit 40 can be controlled by a user, for example, through a user interface 42. Being primarily intended to be distributed to the general population, detector 30, and particularly user interface 42 is preferably “user friendly.” This can be achieved by designing user interface 42 in a manner such that the number of actions which are required from the user to guaranty full functionality of the unit is minimal, preferably one action. Additionally, user interface 42 preferably comprises visual, auditory and/or tactile accessories. User interface 42 may be manufactured in several versions, depending on the nationality and age of the user. For example, different versions of user interface 42 may be labeled in different languages. For children, user interface 42 preferably displays simple instructions which may be transmitted interactively via a communications system.

Besides allowing activation of detector 30, user interface 42 can also provide the user with instructions or information. The information is preferably in a form of sensible signals (visual signals, audio signals, vibrations, etc.) and it may include presence, level or absence of harmful agent, operative status and/or impermeability level of detector 30.

For example, user interface 42 can generate a first sensible signal when power source 38 is operational, a second sensible signal when power source 38 is not operational, a third sensible signal when the components of detector 30 are sealed, a fourth sensible signal when the sealing of one or more components of detector 30 is broken, a fifth sensible signal for a hardware failure, a sixth sensible signal for a software failure and the like. Optionally and preferably, user interface 42 is powered by an additional power source, hence being operative when power source 38 is not operative or during replacement of power source 38.

According to a preferred embodiment of the present invention the level of the sensible signal is selected to allow sensation of the signal at large distances. This can be done, for example, by integrating a powerful light emitting diode in user interface 42. Powerful light emitting diodes are commercially available and can produce light beam to a distance of over 4 km. Alternatively, user interface 42 may comprise powerful audio units, such as commercially available personal defense audio units, also known as “rape alarms.”

The detection information provided by user interface 42 can be in a form of a color code. For example, a first color (say, red) can indicates that the detected agent is hazardous in a certainty level which is above a predetermined threshold (e.g., about 50%), a second color (say, yellow) can indicates that the detected agent is hazardous in a certainty level which is below the threshold, and a third color (say, green) can indicates that the detected agent is not hazardous.

According to a preferred embodiment of the present invention detector 30 may comprise a supplementary sensing unit 56 which preferably continuously monitor environmental conditions. This embodiment is particularly useful when detector 30 operates under a triggering or cueing mode. Hence, when the environmental conditions monitored by unit 56 meet a predetermined set of criteria (e.g., a preliminary detection of a potentially harmful agent), unit 56 generates a signal to activation unit 40 to activate detector 30. The advantage of this embodiment is that triggering or cueing mode improves the reliability and detection performance of detector 30. Detector 30 may comprise more than one supplementary sensing unit for monitoring different environment conditions.

Hence, in one embodiment, one supplementary sensing unit includes a continuous air sampler coupled to a detector (e.g., a visible light laser) which monitors changes in airborne particle characteristics (such as number and size). Upon realization that such changes occur, additional confirmation may be obtained by additional sensing unit (for example, a laser source) that may provide presumptive information as to the possible source (biological or mineral) of the particulate matter. If the combined information from the two sensing unit correlates to a potentially harmful agent, a full operation of detector 30 (e.g., via activation of wetware 34) is initiated.

In another embodiment, supplementary sensing unit 56 comprises a vital signs measuring unit, for measuring vital signs (heart rate, blood pressure, breathing rate, etc.) of the carrier of detector 30 as further detailed hereinabove. When the vital signs of meet a predetermined set of criteria (e.g., are reduced below a predetermined threshold) a signal is transmitted to activation unit 40 to activate detector 30.

Detector 30 may further comprise an atmospheric condition measuring unit 58 for measuring atmospheric conditions, such as, but not limited to, temperature, barometric pressure, solar radiation, wind speed and direction and relative humidity. As stated this information can be used to predict a propagation path of the detected agent.

Detector 30 is preferably encapsulated in an armored encapsulation 62, which is at least partially impermeable and capable of withstanding extreme thermal and/or mechanical conditions. Specifically, encapsulation 62 protects detector 30 from damage due to low temperatures, high temperatures, high humidity, large dust load, extensive rocking and the like. For example, in situations in which detector 30 is deployed on water, encapsulation 62 preferably provides detector 30 with buoyancy and ensures a safe landing without damage.

According to a preferred embodiment of the present invention detector 30 comprises a communication unit 44, for transmitting signals over the communication network. The signals can represent detection information (e.g., presence, level or absence of harmful agents), vital signs, atmospheric conditions and the like. Additionally, the signals can represent an identification code of detector 30, so as to allow the receiving party to identify the source of the signals. As stated, the detection information is preferably combined with location information. Thus, according to a preferred embodiment of the present invention detector 30 comprises a positioning unit 48, for determining the location of detector 30. Positioning unit 48 can operate according to any of the above techniques. The location information is preferably transmitted by communication unit 44 over the communication network, for example, to allow the central location to cross check the detection information of several detectors and/or to identify clustering, as further detailed hereinabove.

Communication unit 44 preferably serves also for receiving signals from the central location or from other detectors in the communication network. According to a preferred embodiment of the present invention signals, received by communication unit 44, control activation unit 40 to remotely activate or select the operational mode of detector 30.

Additionally, the central location can transmit signals to detector 30 so as to unilaterally communicate with the user, for example, via a user display 46. Bilateral communication is also contemplated by allowing transmission of response signals from communication unit 44 to the central location. To further facilitate information exchange between detector 30 and the communication network, detector 30 preferably comprises an image capturing unit 50 (e.g., a stills or video camera) and an input-output audio unit 52 (e.g., a loudspeaker and a microphone).

According to a preferred embodiment of the present invention communication unit 44 is supplemented with at least one communication protocol, tangibly embodied in a readable memory. The communication protocol is preferably configured to allow a takeover of the communication network. This is particularly useful when a harmful agent is detected in a facility which is equipped with an internal communication network, for example, sky-scrapers, shopping malls, governmental building and the like. Upon a detection event in such a facility, the central location can selectively transmit a permission signal to one or more detectors which are carried by key security personnel, which permission signal instructs activation unit 40 to select a takeover mode of detector 30. Once the takeover mode is selected each individual of the security personnel can transmit appropriate messages or instructions to the population via the internal communication network. The takeover mode can also be selected manually, or automatically in case of a detection event, as desired.

Detector 30 can be incorporated in a detection kit which can be released into the population of interest during or prior to initiation of a diffuse signal. Hence, according to another aspect of the present invention there is provided a portable detection kit 70, for releasing into the environment.

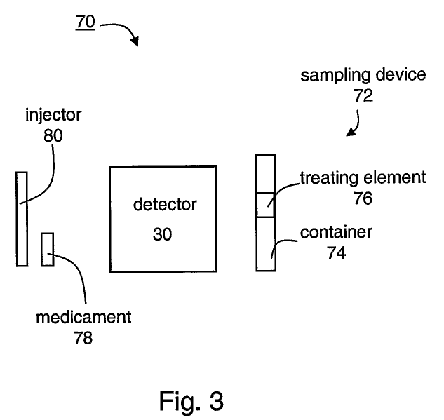

Reference is now made to FIG. 3, which is a schematic illustration of kit 70. Kit 70 preferably comprises one or more sampling devices 72, for selectively sampling environmental material, and an agent detector, which can be similar to detector 30 or a variant thereof. In use, sampling device 72 samples the environmental materials and feeds the sampled materials into detector 30 which senses and analyses the materials as further detailed hereinabove. Optionally and preferably, sampling device 72 treat the sampled material prior to the feeding into detector 30 to enhance the detection accuracy.

Sampling device 72 is preferably designed and constructed to be used more than one time, more preferably a plurality of times. According to a preferred embodiment of the present invention sampling device is adapted to sample environmental fluids (gases or liquids), or solids (e.g., particulates and the like). Thus, sampling device may be provided in a form of a syringe having a syringe needle, which can be operated, as commonly known in the art, to sample materials from the environment by forming an under-pressure within the syringe relative to the environmental pressure.

Sampling device 72 can be adapted either to a continuous sampling or to a single-batch sampling. A particular feature of the present embodiment is the ability of sampling device 72 to treat the sampled materials prior to the feeding into detector 30. Hence, sampling device 72, preferably comprises container 74 and at least one treating element 76 for treating said environmental materials in container 74. Many treating element are contemplated, including, without limitation a filter, an enriching unit, an elution unit, a heating unit, an irradiation unit, a labeling unit, a separating column a sorter, a biological material and the like.

Thus, treating element(s) 76 can be used to perform many treatments which preferably optimize the detection performance of detector 30 in terms of speed, specificity and reliability. The treatments can be chemical and/or physical treatments, including, without limitation, screening, filtration, adsorption, desorption, elution, concentration, chemical reactions, heating and labeling. The treatment can also be used to ensure that the sample is delivered to detector 30 at an appropriate phase, for example, airborne gas, liquid-dissolved gases, liquid-suspended particulates and the like.

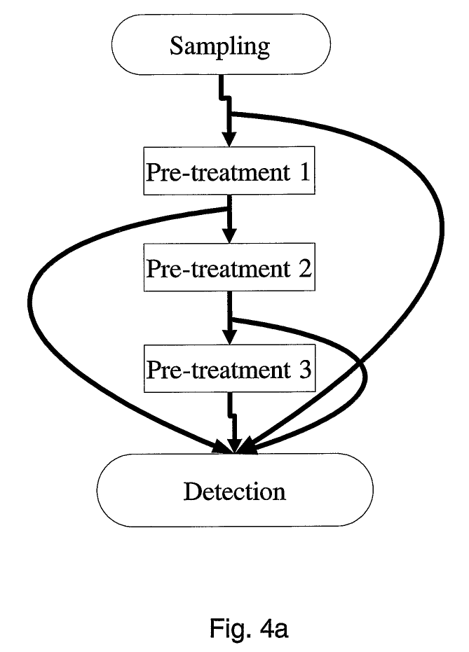

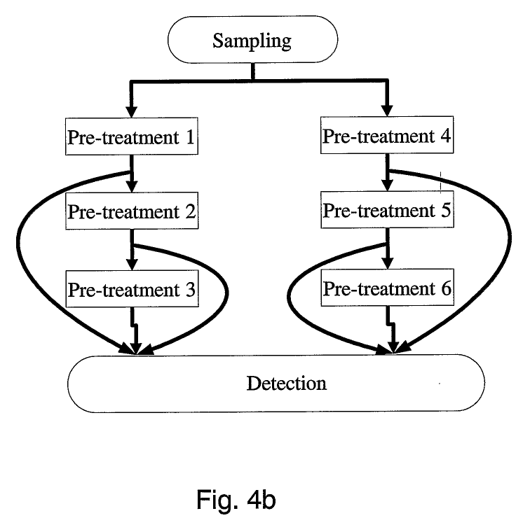

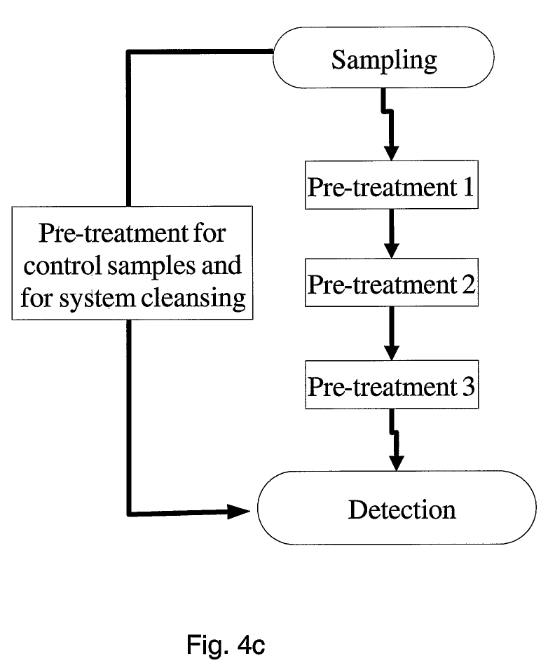

Reference is now made to FIGS. 4a-c, which are flowchart diagrams of several general treatment scenarios which can be employed by kit 70.

Hence, According to a preferred embodiment of the present invention sampling device 72 can perform more than one treatment, either sequentially (FIG. 4a) or contemporaneously (FIG. 4b) as desired. Additionally, sampling device 72 can perform the treatment(s) on a portion or the entire samples and to perform different treatments to different portions of the sample. According to a preferred embodiment of the present invention the treated sample or portion thereof can be transferred to detector 30 in any steps of the treating process, if desired. This embodiment can be used, for example, to skip one or more of the treating steps or to compare detection results obtained after different numbers of treating steps (see FIG. 4c).

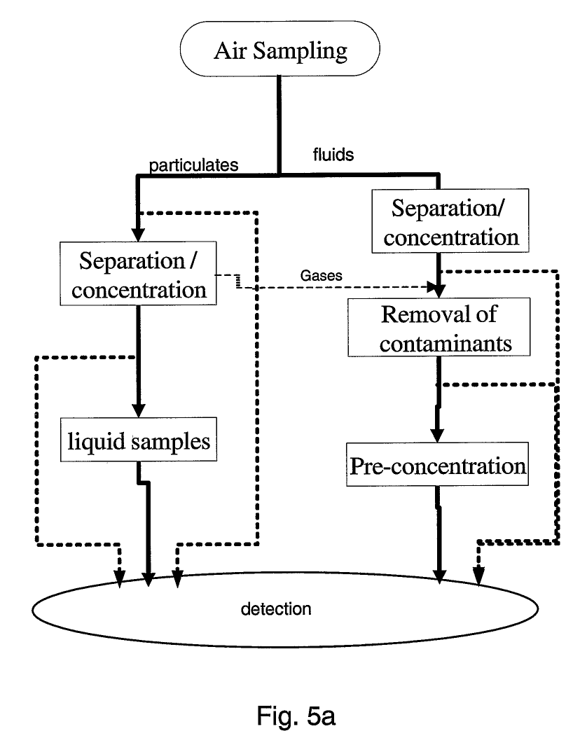

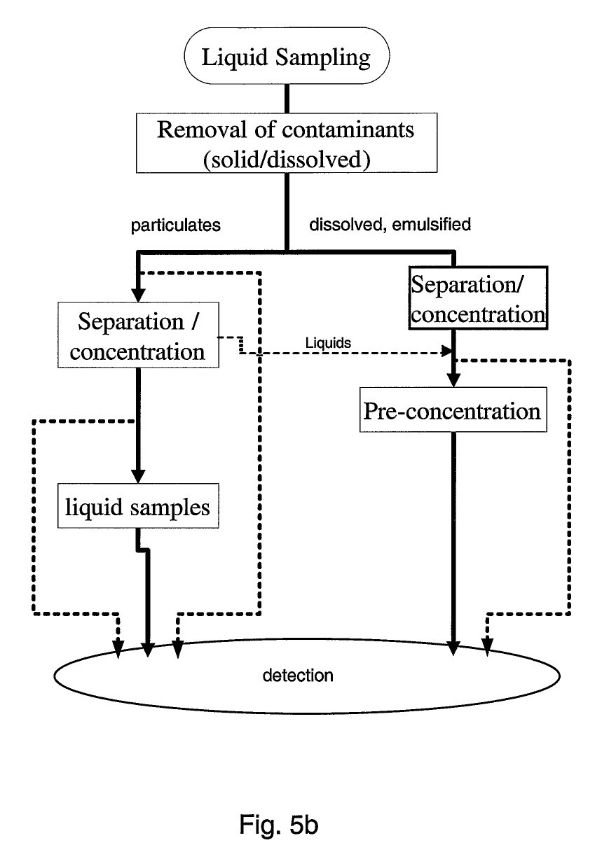

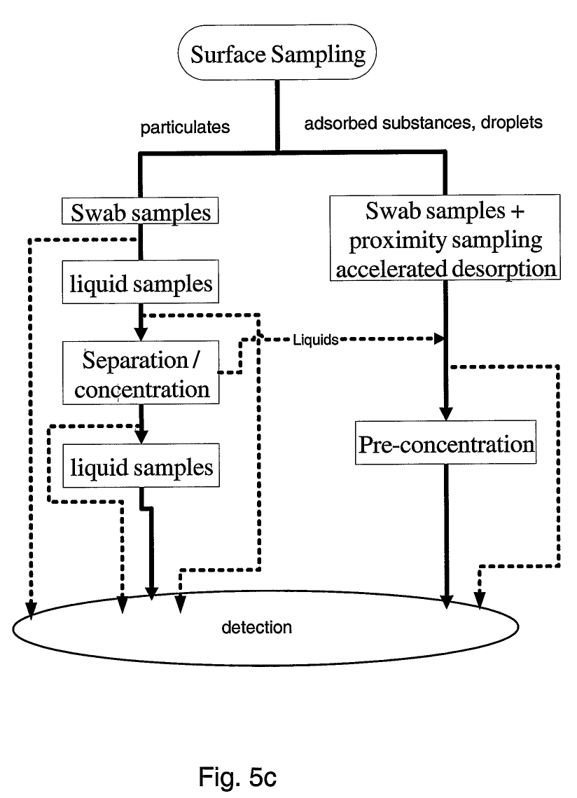

Reference is now made to FIGS. 5a-c, which are flowchart diagrams of treatment scenarios which can be employed, for sampling of air (FIG. 5a), liquid (FIG. 5b) and surface (FIG. 5c), according to a preferred embodiment of the present invention.

When kit 70 is used for air sampling (see FIG. 5a) for airborne toxic chemicals, contemporaneous treatments are preferably performed on particulates (typically biological and radioactive agents) and fluids (typically chemical and radioactive agents). Large volumes of air may be passed through conduits tailored to specifically adsorb target chemicals (for example by using appropriate polymer matrixes). The chemicals can then be desorbed from the adsorbing matrix (e.g., by the application of heat) so as to provide detector 30 with an enriched sample. Similarly, when kit 70 is used for monitoring air for the presence of potentially pathogenic airborne particles, the particles may be concentrated by means of passing the sampled air through impactors (cascade or virtual), cyclones and the like, which provide an enriched sample. The particles can be directly analyzed by detector 30 or, more preferably, transferred to small volumes of liquids for further analysis. The dislodging of particles from the impactors may be achieved by electrostatic, acoustic or any other appropriate method.

When kit 70 is used for liquids sampling (see FIG. 5b), a removal of solid or dissolved contaminates is preferably followed by contemporaneous treatments performed on particulates (typically biological and radioactive agents) and dissolved, suspended or emulsified substances (typically chemical and radioactive agents).

When kit 70 is used for surface sampling (see FIG. 5c), contemporaneous treatments are preferably performed on particulates (typically biological and radioactive agents) and adsorbed substances and droplets (typically chemical and radioactive agents).

For biological materials, the treatments preferably comprise one or more nucleic acid amplification procedures, such as, but not limited to, polymerase chain reaction (PCR), ligase chain reaction (LCR), strand displacement amplification (SDA) and self-sustained sequence replication (3SR). In this embodiment, a number of additional steps may be implemented, for example, lysis of target cells by appropriate solutes, purification of the lysates, addition of reagents and application of temperature cycles.

Referring again to FIG. 3, according to a preferred embodiment of the present invention kit 70 can comprise at least one medicament 78 and optionally an injector 80 for injecting medicament 78. Medicament(s) 78 are preferably selected according to the expected threat so as to allow self-treatment of the user in case of injury. Injector 80 is preferably is easy to carry and safe to use. More particularly, injector 80 is preferably useful for carrying medicaments such as atropine, lidocaine, heart medication, allergy medication and the like. Injector 80 can comprise a safety guard to protect from accidental actuation. According to a preferred embodiment of the present invention injector 80 comprises an automatic needle which penetrates into the muscles of the user upon activation of a suitable mechanism. Such injectors are known in the art and are found, e.g., in U.S. Pat. Nos. 6,530,904, 6,758,110 and 5,968,015.

The kit of the present embodiment may, if desired, be presented in a pack or dispenser device, such as an FDA approved kit, which may contain one or more units of the kit of the present embodiment. The pack may be accompanied by instructions for use. The pack may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or food supplements of an approved product insert.

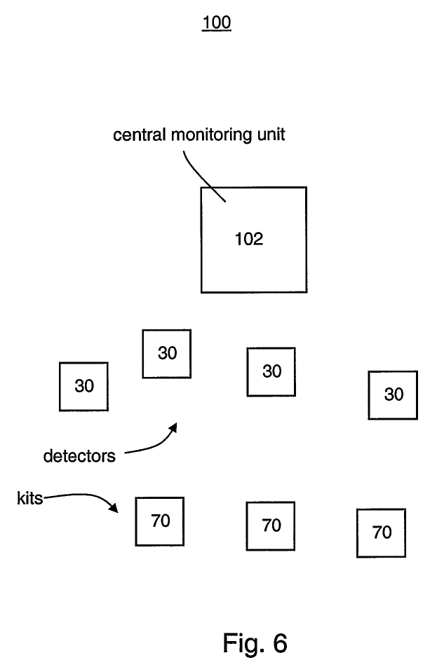

Reference is now made to FIG. 6, which is a schematic illustration of a distributed detection system 100, which can be used for detecting the presence of harmful agents in the environment, according to a preferred embodiment of the present invention. System 100 can employ a plurality of detectors, e.g., detector 30, and/or detection kits, e.g., kit 70, which are preferably placed on vectors in the environment.

According to a preferred embodiment of the present invention system 100 comprises a central monitoring unit 102 which communicates with the plurality of detectors over a communication network as further detailed hereinabove. The detectors can also inter-communicate thereamongst, if desired, for example, to alert neighboring vectors in case of a detection event. Central monitoring unit 102 is preferable located at the aforementioned central location and perform various tasks therefrom. For example, unit 102 can remotely activate detector 30, monitor detection and location data transmitted by detectors 30, cross check detection information of several detectors, identify clustering, communicate with the vectors carrying the detectors and provide permissions to selective detectors in the environment to takeover a local communication network, as further detailed hereinabove.

Any of the above embodiments can be used to detect or identify many agents, including, without limitation, chemical warfare agents, toxic industrial chemicals, explosives, narcotic chemicals, biological agents and radioactive agents.

Specifically to chemical warfare agents, the following agents are contemplated: nerve blocking agents (e.g., tabun, methylphosphonothioic acid, sarin and soman), blister inducing agents (e.g., sulphur mustard, nitrogen mustard, distilled mustard, mustard lewisite, lewisite, phosgene oximine, ethyldichloroarsine and methyldichloroarsine), choke inducing agents (e.g., phosgene, diphosgene, chlorine and chloropicrin), vomiting inducing agents (e.g., diphenyl-dichloroarsine, adamsite and diphenylcyanoarsine) blood destructing agents (e.g., hydrogen cyanide, cyanogen chloride and arsine) and other chemical warfare agents.

Specifically to biological agents the following agents or type of agents are contemplated: viruses, crimean-congo haemorrhagic fever virus, eastern equine encephalitis virus, ebola viruses, equine morbillivirus, lassa fever virus, marburg virus, rift valley fever virus, south american haemorrhagic fever viruses (junin, machupo, sabia, flexal, guanarito), tick-borne encephalitis complex viruses, variola major virus (smallpox virus), venezuelan equine encephalitis virus, viruses causing hantavirus pulmonary syndrome, yellow fever virus, bacteria, bacillus anthracis, brucella abortus, b. melitensis, b. suis, burkholderia (pseudomonas) mallei, burkholderia (pseudomonas) pseudomallei, clostridium botulinum, francisella tularensis, yersinia pestis, coxiella burnetii, rickettsia prowazekii, rickettsia rickettsii, fungi, coccidioides immitis, toxins, abrin, aflatoxins, botulinum toxins, clostridium perfringens epsilon toxin, conotoxins, diacetoxyscirpenol, ricin, saxitoxin, shigatoxin, staphylococcal enterotoxins, tetrodotoxin, t-2 toxin, protozoa, cryptosporidium, giardia.

Specifically to radioactive agents the following agents are contemplated: radioactive cobalt agents, such as, but not limited to, various isotopes of cobalt, cesium, plutonium, uranium, radium, radon, americium, polonium, bismuth, thorium and the like.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of detecting the presence of harmful agents in the environment comprising:
providing a plurality of portable agent detectors,
placing said detectors on vectors in said environment,
connecting said detectors over a communication network,
upon detection of an agent by a first detector, signaling detectors identified as being nearby to carry out corresponding agent detection tests,
thereby to confirm or localize an initial agent detection.

2. The method of claim 1, further comprising monitoring said detectors at a central location via said communication network.

3. The method of claim 2, wherein said signaling said nearby detectors comprises remotely activating said nearby detectors.

4. A method of detecting the presence of harmful agents in the environment comprising:
providing a plurality of portable agent detectors able to generate a detection event in the presence of any of said harmful agents,
placing said detectors on vectors in said environment,
connecting said detectors over a communication network,
at a central location monitoring said detectors to identify clustering of said detection events, said clustering being to confirm or localize an initial agent detection.

5. The method of claim 4, further comprising, upon a detection event generated by a first detector, signaling detectors identified as being nearby to said first detector, to carry out corresponding agent detection tests.

6. The method of claim 2 further comprising, upon a detection event generated by a first detector, alerting vectors identified as being nearby to said first detector.

7. A method of detecting the presence of harmful agents, comprising:
providing a plurality of portable agent detectors able to generate a detection event in the presence of any of said harmful agents,
associating said detectors with cellular communication units to allow central monitoring of said detectors, and
placing said detectors and cellular communication units on vectors in said environment.

8. The method of claim 7, further comprising associating said detectors with positioning units, and monitoring locations of individual detectors using a positioning network.

9. The method of claim 2 further comprising obtaining atmospheric conditions at a location of said first detector and using said atmospheric condition to predict a propagation path of said agent.

10. A portable agent detector for releasing into an environment to detect harmful agents released into said environment, the agent detector comprising:
a removable sensing cassette comprising sensing wetware; and
a processing unit for processing signals from said sensing cassette.

11. The portable agent detector of claim 10, further comprising an activation unit for activating or selecting an operational mode of the portable agent detector.

12. The portable agent detector of claim 11, further comprising a user interface, wherein said activation unit is controllable by said user interface.

13. The portable agent detector of claim 10, further comprising an atmospheric condition measuring unit for measuring at least one atmospheric condition.

14. A method for analyzing a diffuse signal from an environment comprising:
releasing a plurality of signal detectors into said environment, each signal detector being able to generate a detection event in the presence of said diffuse signal;
monitoring each of said signal detectors over a communication network for said detection events and for current location data of said signal detectors; and
identifying clustering of said detection events as an indication of the presence of said diffuse signal.

15. The method of claim 14, further comprising, upon a detection event generated by a first detector, signaling detectors identified as being nearby to said first detector, to carry out corresponding detection tests.

16. The method of claim 14, further comprising, upon a detection event generated by a first detector, generating alert signals and transmitting said alert signals to detectors identified as being nearby to said first detector.

17. A method for analyzing a diffuse signal from an environment comprising:
releasing a plurality of signal detectors into said environment, each signal detector being able to generate a detection event in the presence of said diffuse signal;
monitoring each of said signal detectors over a communication network for said detection events and for current location data of said signal detectors;
upon identifying a detection event, checking with other nearby detectors for confirmation of said diffuse signal.

18. The method of claim 17, further comprising generating alert signals and transmitting said alert signals to said nearby detectors.

19. The method of claim 17, wherein said checking with said nearby detectors comprises transmitting instructions to vectors carrying said nearby detectors to locally activate said nearby detectors.

20. The method of claim 17, wherein said checking with said nearby detectors comprises remotely activating said nearby detectors.

21. The method of claim 3 further comprising identifying clustering of said detection events.

22. The method of claim 14 further comprising obtaining atmospheric conditions at a location of at least one detector and using said atmospheric condition to predict a propagation path of the diffuse signal.

23. A distributed detection system for detection of the presence of harmful agents in an environment, comprising:
a central monitoring unit; and
a plurality of portable agent detectors enabled for communication with said central monitoring unit and configured for producing detection events in the presence of said harmful agents and communicating said detection events to said central monitoring unit; said portable agent detectors being mounted on mobile vectors for release into said environment.

24. The system of claim 23, wherein at least one of said plurality of portable agent detectors comprises:
a sensing unit having an operative mode and a non-operative mode;
an activation unit being in communication with a central location, for selecting between said operative mode and said non-operative mode; and
a processing unit for processing signals from said sensing unit.

25. The system of claim 24, wherein said sensing unit and said processing unit are designed and constructed to provide detection information within predetermined and variable time periods.

26. The system of claim 24, wherein at least one of said plurality of portable agent detectors further comprises a user interface, wherein said activation unit is controllable by said user interface.

27. A portable agent detector for releasing into an environment to detect harmful agents released into said environment, the agent detector comprising:
a sensing unit having an operative mode and a non-operative mode;
an activation unit being in communication with a central location, for selecting between said operative mode and said non-operative mode; and
a processing unit for processing signals from said sensing unit.

28. The portable agent detector of claim 27, wherein said sensing unit and said processing unit are designed and constructed to provide detection information within predetermined and variable time periods.

29. The portable agent detector of claim 27, further comprising a user interface, wherein said activation unit is controllable by said user interface.

30. The portable agent detector of claim 27, further comprising an atmospheric condition measuring unit for measuring at least one atmospheric condition.

31. A portable agent detector for releasing into an environment to detect harmful agents released into said environment, the agent detector comprising:
a sensing unit for detecting presence or absence of harmful agents;
a processing unit for processing signals from said sensing unit;
a positioning unit for determining a location of the agent detector; and
a communication unit for transmitting said location and signals representing presence or absence of harmful agents over a communication network.

32. The portable agent detector of claim 31, further comprising an atmospheric condition measuring unit for measuring at least one atmospheric condition, and further wherein said communication unit is operable to transmit said at least one atmospheric condition over said communication network.

33. The portable agent detector of claim 31, being identifiable by an identification code, and further wherein said communication unit is operable to transmit said identification code over said communication network.

34. The portable agent detector of claim 31, wherein said sensing unit and said processing unit are designed and constructed to provide detection information within predetermined and variable time periods.

35. The portable agent detector of claim 31, further comprising an activation unit for activating or selecting an operational mode of the portable agent detector.

36. A portable vital signs detector to detect vital signs of a mammal the portable vital signs detector comprising:
a sensing unit for detecting the vital signs of the mammal;
a processing unit for processing signals from said sensing unit;
a positioning unit for determining a location of the vital signs detector; and
a communication unit for transmitting said location and signals representing the vital signs.

37. A personal accessory device having an agent detector to detect harmful agents released into said environment, the agent detector comprising:
a sensing unit having an operative mode and a non-operative mode;
an activation unit being in communication with a central location, for selecting between said operative mode and said non-operative mode; and
a processing unit for processing signals from said sensing unit.

38. The personal accessory device of claim 37, wherein said activation unit is capable of deactivating any functioning unit of the personal accessory device other than said sensing unit and said processing unit.

39. The personal accessory device of claim 37, wherein said sensing unit and said processing unit are designed and constructed to provide detection information within predetermined and variable time periods.

40. The personal accessory device of claim 37, further comprising a user interface, wherein said activation unit is controllable by said user interface.

41. The personal accessory device of claim 37, further comprising an atmospheric condition measuring unit for measuring at least one atmospheric condition.

42. A personal accessory device having an agent detector to detect harmful agents released into said environment, the agent detector comprising:
a sensing unit for detecting presence or absence of harmful agents;
a processing unit for processing signals from said sensing unit;
a positioning unit for determining a location of the agent detector; and
a communication unit for transmitting said location and signals representing presence or absence of harmful agents over a communication network.

43. The personal accessory device of claim 42, further comprising an atmospheric condition measuring unit for measuring at least one atmospheric condition, and further wherein said communication unit is operable to transmit said at least one atmospheric condition over said communication network.

44. The personal accessory device of claim 42, being identifiable by an identification code, and further wherein said communication unit is operable to transmit said identification code over said communication network.

45. The personal accessory device of claim 42, wherein said sensing unit and said processing unit are designed and constructed to provide detection information within predetermined and variable time periods.

46. The personal accessory device of claim 42, further comprising an activation unit for activating or selecting an operational mode of the agent detector.

47. The portable agent detector of claim 10 further comprising detection hardware.

48. The portable agent detector of claim 47, wherein said detection hardware comprises optical detection hardware.

49. The portable agent detector of claim 11, further comprising vital signs measuring unit for measuring vital signs of a mammal carrying the agent detector.

50. A portable detection kit for releasing into an environment to detect harmful agents released into said environment, the portable detection kit comprising:
at least one sampling device for selectively sampling environmental materials; and
an agent detector having a removable sensing cassette comprising sensing wetware, and a processing unit for processing signals from said sensing wetware.

51. The portable detection kit of claim 50, wherein said at least one sampling device is reusable.

52. The portable agent detector of claim 11 wherein said activation unit is in communication with a central location and being controllable thereby, hence allowing a remote activation or operational mode selection of said agent detector.

53. The portable agent detector of claim 11 further comprising a supplementary sensing unit capable of continuously monitoring environmental conditions, and generating a signal to said activation unit to activate said agent detector when said environmental conditions meet a predetermined set of criteria.

54. The portable agent detector of claim 53, wherein said predetermined set of criteria comprises preliminary detection of a potentially harmful agent.

55. The portable agent detector of claim 10 further comprising a communication unit, for transmitting signals representing presence, level or absence of harmful agents over a communication network.

56. The portable agent detector of claim 55, being identifiable by an identification code, and further wherein said communication unit is operable to transmit said identification code over said communication network.

57. The portable agent detector of claim 55, wherein said agent detector further comprises a positioning unit, for determining a location of the agent detector, wherein said communication unit is operable to transmit said location over said communication network.

58. The portable agent detector of claim 10 wherein said agent detector further comprises an automatic sampling unit for automatically sampling fluids from the environment, and transferring said fluids to said sensing unit.

59. The portable agent detector of claim 10 further comprising an image capturing unit.

60. The portable agent detector of claim 10 further comprising an input-output audio unit.

61. The portable agent detector of claim 55, wherein said communication unit is supplemented with at least one communication protocol, tangibly embodied in a readable memory, said at least one communication protocol being configured to allow a takeover of said communication network.

62. A sampling device for selectively sampling environmental materials, the sampling device comprising a sampling element, a container and at least one treating element for treating the environmental materials in said container.

63. A method of determining presence or absence of harmful agents in the environment, comprising:
providing a plurality of portable agent detectors having an operative mode and a non-operative mode;
selecting non-operative modes for at least a portion of said plurality of detectors, and placing said plurality of detectors on vectors in the environment;
connecting said plurality of detectors over a communication network having a plurality of base stations;
activating detectors associated with at least one base station to carry out agent detection tests; and
receiving information from said activated detectors, thereby determining presence or absence of harmful agent in a region defined by said activated detectors.

64. The method of claim 63, further comprising obtaining atmospheric conditions at said region and using said atmospheric condition to predict a propagation path of said agent.

65. The method of claim 1 wherein said vectors are mammals.

66. The method of claim 65, further comprising measuring vital signs of said mammals.
